(19) United States
(12) Patent Application Publication
(10) Pub. No.: US 2025/0387112 A1
(43) Pub. Date: Dec. 25, 2025

(54) Soft Tissue Cuff

(71) Applicant: Stryker Corporation, Portage, MI (US)

(72) Inventors: Joseph L. Mark, Indianapolis, IN (US); Brian C. Dougherty, Terre Haute, IN (US)

(73) Assignee: Stryker Corporation, Portage, MI (US)

(21) Appl. No.: 19/244,434

(22) Filed: Jun. 20, 2025

Related U.S. Application Data
(60) Provisional application No. 63/661,958, filed on Jun. 20, 2024.

(51) Int. Cl. A61B 17/02; A61B 34/20

(52) U.S. Cl. CPC A61B 17/0218; A61B 17/0293; A61B 34/20; A61B 2017/0225

(57) ABSTRACT

A soft tissue cuff apparatus for subcortical surgery of the brain includes a cuff body. The cuff body forms a ring-like shape in an expanded position and is formed of a flexible material. The cuff body extends between a first lip and a second lip, thereby forming a concave outer periphery where, during use, the cuff body is configured to contact and cradle at least a portion of the cortex. The first and second lips are configured to form a hold on to the cortex such that the soft tissue cuff apparatus creates a stable fit within the cortex to form a surgical corridor and allow access to a surgical site under the cortex without subjecting the surrounding soft tissue to further manipulation.

### CROSS REFERENCE TO RELATED APPLICATIONS

This application claims the benefit of U.S. Provisional Patent Application No. 63/661,958 filed on Jun. 20, 2024, which is hereby incorporated by reference in its entirety.

### TECHNICAL FIELD

The present disclosure relates generally to a soft tissue cuff of a surgical access system for use with delicate and critical tissues.

### BACKGROUND

Diagnosis and treatment of conditions affecting the brain are among the most difficult and complex problems that face the medical profession. The brain is a complex and delicate soft multi-component tissue structure that controls bodily functions through a complex neural network connected to the rest of the body through the spinal cord. The brain and spinal cord are contained within and protected by significant bony structures, e.g., the skull and the spine. Given the difficulty of accessing the brain through the hard bony protective skull and the delicate network and complex interactions that form the neural communication network contained within the brain that define the human body's ability to carry on its functions of speech, sight, hearing, functional mobility, reasoning, emotions, respiration and other metabolic functions, the diagnosis and treatment of brain disorders presents unique challenges not encountered elsewhere in the body.

Abnormalities such as intracranial cerebral hematomas (ICH), abscesses, glioblastomas (GB), metastases (mets) and functional diseases manifest themselves in the intraparenchymal subcortical space (i.e., the white matter) of the brain are particularly challenging to access, let alone treat. The brain contain eloquent communication structures (neural network) which are located in the subcortical space which extend up and into the cortex, called fiber tracts and fascicles. Thus, traditionally, unless the ICH, GB, and/or mets (subcortical abnormalities) presented other than superficial, access is considered challenging to achieve without causing transient or permanent deficits during access. Similarly, tissue abnormalities such as tumors, cysts and fibrous membrane growths which manifest within the intraventricular space of the brain are considered challenging to safely access without the same risks. Because of this deeply seated subcortical abnormalities are often termed inoperable due to their locations.

In order to operate surgically on and in the brain, access must be obtained through the skull and delicate brain tissue containing blood vessels and nerves that can be adversely affected by even slight disturbances. Therefore, great care, before, during, and after a procedure, must be taken so as not to disturb delicate blood vessels and nerves to prevent adverse consequences resulting from a surgical intervention.

### BRIEF DESCRIPTION OF THE DRAWINGS

The embodiments of the present disclosure are pointed out with particularity in the appended claims. However, other features of the various embodiments will become more apparent and will be best understood by referring to the following detailed description in conjunction with the accompanying drawings in which:

FIG. 1 illustrates a perspective view of an example soft tissue cuff in an expanded position;

FIG. 2 illustrates the soft tissue cuff of FIG. 1 and a surgical tool;

FIG. 3 illustrates the soft tissue cuff of FIG. 1 in a folded position;

FIG. 4 illustrates another example soft tissue cuff in a resting state.

FIG. 5 illustrates the soft tissue cuff of FIG. 4 in an insertion state;

FIG. 6 illustrates another example soft tissue cuff in a resting state;

FIG. 7 illustrates a bottom perspective view of the soft tissue cuff of FIG. 6;

FIG. 8 illustrates a perspective view of a obturator assembly;

FIG. 9 illustrates a perspective view of an obturator of FIG. 8 in an inserted state;

FIG. 10A illustrates a perspective view of an obturator in an expanded state;

FIG. 10B illustrates a perspective view of the obturator of FIG. 10A in a collapsed state;

FIG. 11A illustrates a perspective view of the obturator of FIG. 10A in an expanded state within the soft tissue cuff;

FIG. 11B illustrates a perspective view of the obturator of FIG. 10A in a collapsed state within the soft tissue cuff;

FIG. 11C illustrates a perspective view of the obturator of FIG. 10A upon removal from the soft tissue cuff;

FIG. 12A illustrates a perspective view of another example obturator in an expanded state;

FIG. 12B illustrates a perspective view of the obturator of FIG. 12A in a collapsed state;

FIG. 13A illustrates a perspective view of the obturator of FIG. 12A in an expanded state within the soft tissue cuff;

FIG. 13B illustrates a perspective view of the obturator of FIG. 12A in a collapsed state within the soft tissue cuff; and

FIG. 13C illustrates a perspective view of the obturator of FIG. 12A upon removal from the soft tissue cuff.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Disclosed herein is a soft tissue cuff configured to be used with a surgical access system that provides surgeons with an enhanced ability to minimize trauma to the patient, while providing efficient, minimally invasive surgical techniques, such as, for example, during intracranial surgical procedures. In the case of intracranial surgery, it would be referred to as a soft tissue cuff or cortical cuff, which may be used for applications of targeted and effective treatment regimens to protect the cortex and its eloquent structures.

During subcortical surgery of the brain, various surgical access systems may be used to access an abnormality in an effort to minimize damage to the blood vessels, cranial nerves, fiber tracts and fascicles, etc. Such surgical access systems may include NICO BrainPath™, Vycor BrainSite™, among other tubular surgical access systems such as blade retractors. The surgical access system may be used to provide access to the abnormality in the subcortical space of the brain. A tubular access device system such as BrainPath™ may include an obturator which may be used to displace tissues of the brain to achieve access to the abnormality and a sheath may be placed within the brain and may remain there after the obturator is removed to permit access to the surgical site. However, even when other types of standard retractors are employed, such as blade retractors it can be desirous to remove diseased tissue which is immediately under the cortex while maintaining the integrity of the overlying cortex.

Often times, the abnormality may extend up to but does not invade the cortex. At the end of a surgical procedure, the retractor is removed to allow for final cleanup of the proximal aspects of the abnormality. However, the abnormality may lie immediately below or under the cortical rim or other soft tissue. Thus, the overlying cortex of the surgical site is manipulated to expose the underlying abnormality of the intra-axial parenchyma. The manipulation of this cortex may cause transient or permanent damage to the cortex or other eloquent structures such as vessels and membranes associated with the cortex. This tissue may then be removed by the surgeon due to an inability to preserve or separate the healthy tissues from the underlying tissue to be removed.

The soft tissue cuff may be used in conjunction with a retractor system or independent of any other retractor system. In the applications where the area of interest is present just under the cortex, it may be used as a retractor system for shallow presenting targets that are areas of interest. However, if the area of interest is deeper into the white matter rather than superficial, the cortical cuff may be used in conjunction with a retractor system such as a tubular retractor thus enabling the protection of the cortex as the standard retractor system is manipulated and repositioned during the procedure thus providing protection to the cortex. Alternately, it may also be inserted into the aperture which was previously created by the standard retractor system and placed within the cortical rim which is surrounding the area immediately above the subcortical surgical site. The soft tissue cuff may be flexible and may allow for manipulation around its radius once placed at the surgical site. The soft tissue cuff may form a ring-like shape made of pliable, and/or malleable, soft, foldable, elastomeric material such as silicone. During placement of the soft tissue cuff, the soft tissue cuff may be pinched, or folded, to enable the diameter of the soft tissue cuff, which may be larger in diameter than the aperture which was created by the surgical access system. Once in place, the soft tissue cuff may resume its normal open relaxed state and form a ring at the surface of the cortex. During the surgical clean up, the soft tissue cuff allows the surgeon to safely manipulate the tissue surrounding the soft tissue cuff without directly touching the surrounding cortical tissue with another instrument thus allowing for the soft manipulation and movement of surrounding soft tissue and the associated eloquent structures of the cortex. Because of the radial shape of the soft tissue cuff, when inside diameter of the soft tissue cuff is manipulated with a surgical tool, the force will be dissipated around the circumferential surface area of the cuff. Without the soft tissue cuff, the surgical tool may cause a single compressive force or even cutting of the cortex to be realized at the tissue, potentially harming the tissue.

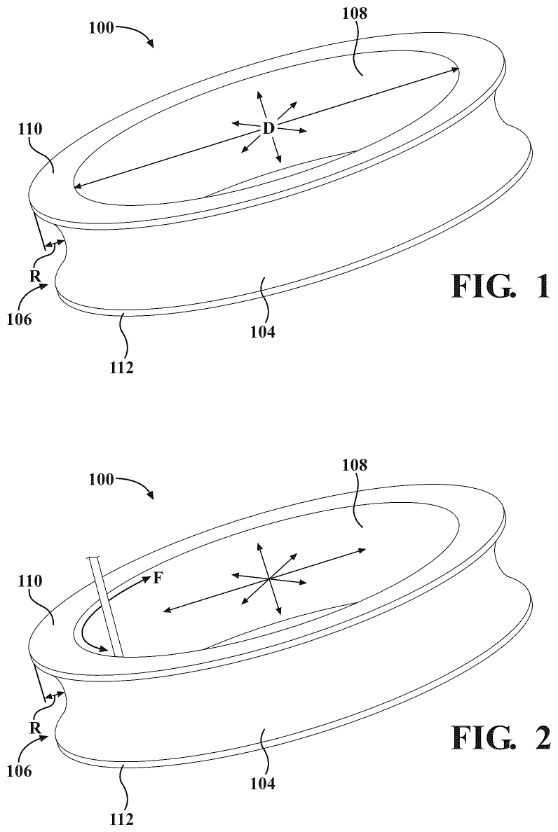

FIG. 1 illustrates an example soft tissue cuff 100 in an expanded position. The soft tissue cuff 100 may include a cuff body 104 configured to form a circular shape. The body 104 may form a concave outer portion 106 between a first lip 110 and a second lip 112. The concave outer portion 106 may be configured to contact the cortex and cradle it. The lips 110, 112 may form a gentle hold on the cortex, creating a stable fit therein so as to prevent the cuff 100 from moving. The concave outer portion 106 may have a radius R. In one example, the radius R may be approximately 4 millimeters.

The cuff 100 may have in inner surface 108 forming an inner diameter D. The diameter D in the expanded position may range in size depending on the procedure, size and/or age of patient, surgical tools being used during the procedure, etc. In one example, the inner diameter may be approximately 17 millimeters. In certain implementations, the cuff is dimensioned with the radius R for the concave outer portion 106 in a size ratio range with the inner diameter D of inner surface 108 of from 1:5 to 1:3. The combination of this ratio range with the material selection of the cuff 100 described below allows the cuff 100 to be sufficiently flexible when the cuff 100 is manipulated during insertion and sufficiently resilient to enable to cuff 100 to maintain the access opening created after insertion.

The cuff 100 may be formed of a flexible and/or soft material such as flexible elastomer, such as medical grades of silicone, rubber, urethane, low density polyethylene, high density polyethylene, PVC or other semi-rigid plastic, etc. The material may have some rigidity such that the cuff 100 may maintain an expanded position without compressing under inward forces created by the surrounding tissues. However, the cuff 100 may be flexible and responsive to direct force such as that created by a surgical tool to protect and buffer the brain tissue from damage or insult. The soft flexible material of the cuff 100 may prevent pinching of the fibers, membranes and vessels of the cortex while maintaining a corridor at the cortex.

Alternatively, the cuff 100 may be made of a low tensile metal material, for example, aluminum which is encapsulated/coated or otherwise covered with a pliable elastomeric material (not shown). Suitable elastomeric materials are described above.

FIG. 2 illustrates an example soft tissue cuff 100 in an expanded position and a surgical tool 120 arranged in the center thereof. The surgical tool 120 may include a tubular retractor, scalpel, or other tool used during the surgical procedure. The surgical tool 120 may be used to further manipulate the tissue after a procedure to further ‘clean up’ the surgical site. In some procedures, residual abnormalities may remain just under the cortex. The surgical tool 120 may move around the surgical cuff 100 to expose the underlying area under the cortex thus allowing for the examination and inspection of this which was under the over lying cortex and then the removal of the abnormality within the confines of the cuff 100. If the surgical tool 120 abuts or comes into contact with the inner surface 108 during the manipulation, the inner surface 108 may be configured to receive the force created by the surgical tool 120 and radially dissipate it, as shown by force F in FIG. 2. By radially dissipating the force F, the tissue adjacent thereto does not realize nor receive a single direct force F, but instead realizes a fraction of the force F created by the surgical tool 120. The force F is dissipated across the soft tissue cuff 100, thus preventing a single pin-pointed force F that could result in tissue damage.

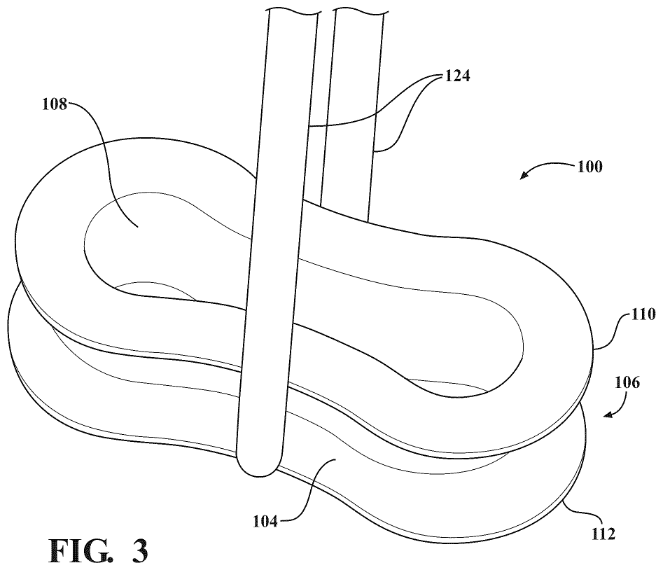

FIG. 3 illustrates the soft tissue cuff 100 in a folded position. As explained above, the soft tissue cuff 100 may be pliable. The soft tissue cuff 100 may be folded, bent, etc., in any number of ways. In one example, prior to installation or placement at the cortex, the soft tissue cuff 100 may be pinched by forceps 124 or other instruments suited for such delivery of the soft tissue cuff 100. The forceps 124 may hold the soft tissue cuff 100 there between and be used for placement of the soft tissue cuff 100 during the procedure. Using the forceps 124, the soft tissue cuff 100 may be guided through the corridor and placed at the cortex. During this process, the soft tissue cuff 100 may be placed at an angle, in addition to being folded, in an effort to fit through the corridor. Upon release of the forceps 124, the soft tissue cuff 100 may resume its normal, circular shape. Once in place, the soft tissue cuff 100 may form a ring at the surface of the cortex giving a surgeon access to the surgical site, as explained above.

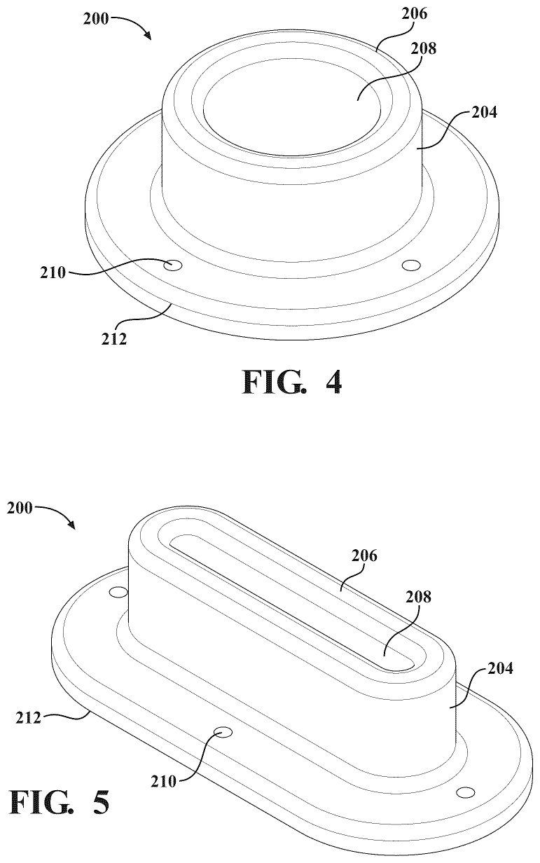

FIG. 4 illustrates another example soft tissue cuff 200 in a resting state. The soft tissue cuff 200 may include a cuff body 204 forming a cylindrical shape. The cuff body 204 may include a rounded end 206 at a distal end of the cuff 200. The cuff body 204 may form in inner surface 208 forming an inner diameter D. The diameter D in the expanded position may range in size depending on the procedure, size and/or age of patient, surgical tools being used during the procedure, etc. In one example, the inner diameter may be approximately 15 millimeters.

The cuff 200 includes a cylindrical base 212 or flange at the opposite end of the rounded end 206. The base 212 may define at least one hole 210. In the example shown in the figures, the at least one hole 210 includes a plurality of holes 210, each being equidistantly spaced from an adjacent one of the holes 210. However, more or less holes may be included, as well as varying spacing around the base 212.

The holes 210 may have several advantages and functions. The holes 210 may allow for the soft tissue cuff 200 to be manipulated. The holes 210 may also be used to receive suture material to allow the soft tissue cuff 200 to be secured to surrounding tissue, such as the dura. Additionally, other device may be used in conjunction with the soft tissue cuff 200, such as a Greenberg style retractor system.

The cuff 200 may be formed of a flexible and/or soft material such as flexible elastomer, such as medical grades of silicone, rubber, urethane, low density polyethylene, high density polyethylene, PVC or other semi-rigid plastic, etc. The material may have some rigidity such that the cuff 200 may maintain an expanded position without compressing under inward forces created by the surrounding tissues. However, the cuff 200 may be flexible and responsive to direct force such as that created by a surgical tool both within the cuff body 204 and external to the cuff 200. The soft flexible material of the cuff 200 may prevent pinching, abrasion of the fibers, membranes and vessels of the cortex while maintaining a corridor at the cortex.

FIG. 5 illustrates the soft tissue cuff 200 of FIG. 4 in an insertion state. In this state, the soft tissue cuff 200 may be manipulated by a surgical tool (not shown in FIG. 5) within the body 204. As explained above with respect to FIG. 2, after insertion of the soft tissue cuff 200, the surgical tool may include a tubular retractor, probe, forceps, tissue removal tools, or other tools and instruments used during the surgical procedure.

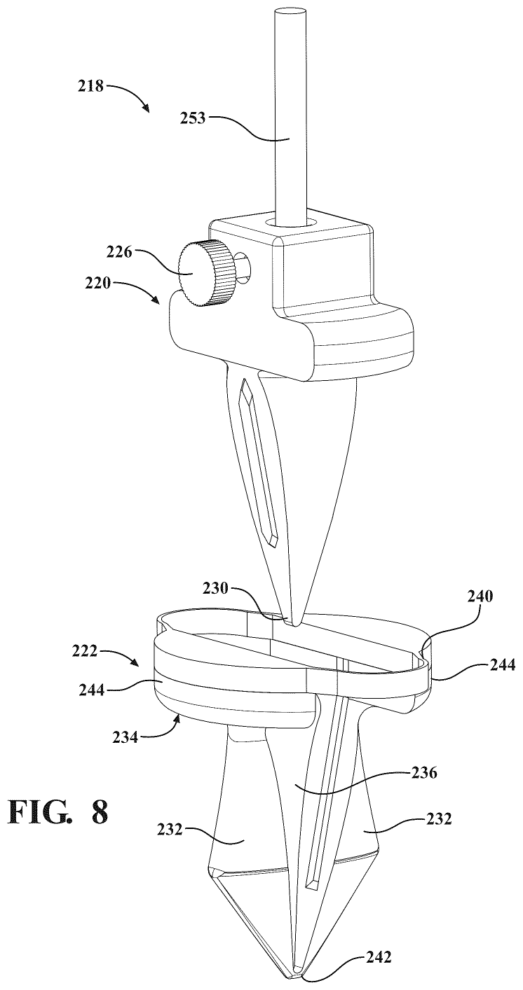

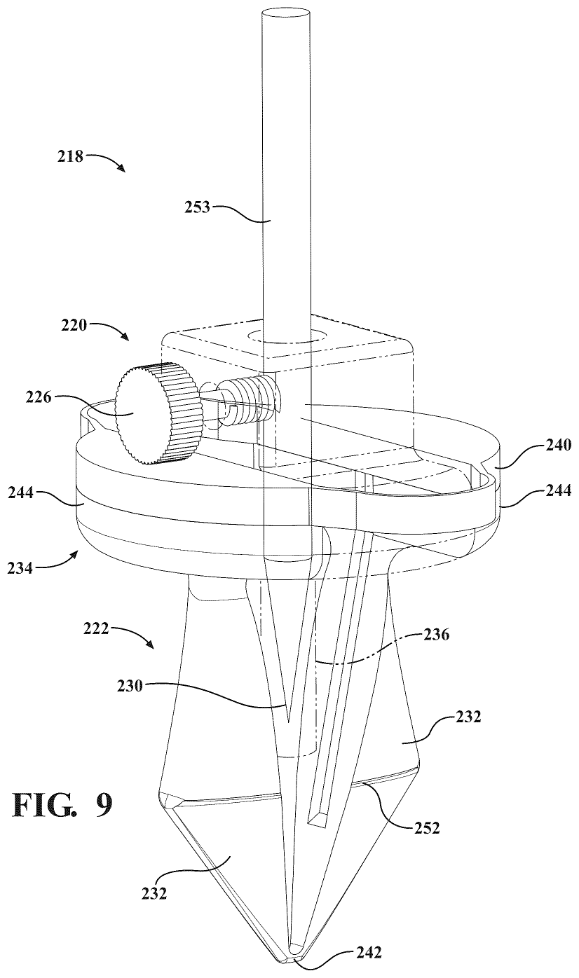

Prior to insertion of the soft tissue cuff 200 at the surgical site, however, the soft tissue cuff 200 may also be manipulated. In the example of FIG. 5, prior to insertion of the soft tissue cuff 200, the tool may be an obturator and the soft tissue cuff 200 may create an insertion profile that mimics the profile of the obturator. Manipulation of the soft tissue cuff 200 prior to installation or placement at the cortex may be the result of the soft tissue cuff 200 being stretched by the obturator or other instruments suited for such delivery at the surgical site. Upon removal of the obturator at the surgical site, the soft tissue cuff 200 may resume its normal, circular shape. Once in place, the soft tissue cuff 200 may form a ring at the surface of the cortex giving a surgeon access to the surgical site, as explained above. This process is explained in more detail with respect to FIGS. 8 and 9.

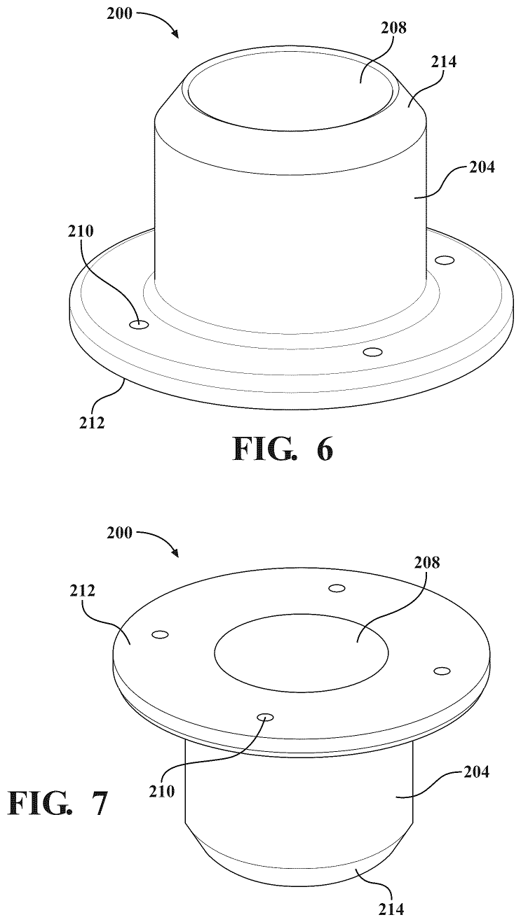

FIG. 6 illustrates another example soft tissue cuff 200 in the resting state. FIG. 7 illustrates a bottom perspective view of the soft tissue cuff 200 of FIG. 6. This example soft tissue cuff 200 may be similar to that of FIG. 4, but may include a tapered end 214. Further, it should be noted that the cuff body 206 may vary in height depending on the example. Such difference in heights may allow for different patient cortex depths, varying instruments to be used, etc. In other words, the height of the tissue cuff 200 may be chosen based on the patient's particular condition, instruments required or preferred by the surgeon, and other factors.

FIG. 8 illustrates a perspective view of an obturator assembly 218 having a navigation device probe adapter 220 and obturator 222. FIG. 9 illustrates a perspective view of the obturator assembly 218 of FIG. 8 in an inserted state.

The obturator assembly 218 may be a surgical access assembly, or part of a surgical access assembly, including the obturator 222. The navigation device probe adapter 220 may be configured to receive a stylet 253, such as a navigation probe, which may be removably retained within the obturator 222. FIG. 8 illustrates an example navigation device probe adapter 220 configured to be received, at least in part, by the obturator 222. The navigation device probe adapter 220 is configured to be inserted within the obturator 222 and a securement arrangement 226 may also be provided. The securement arrangement 226 is configured to operatively retain a stylet 253 within the navigation device probe adapter 220. The stylet 253 may define a distal tip 230 extending beyond the obturator 222.

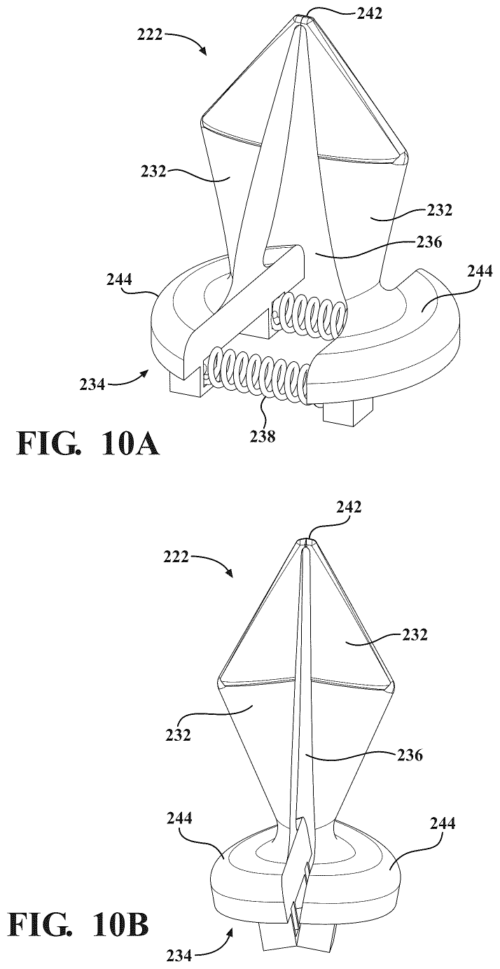

The obturator 222 is discussed in more detail below with respect to FIGS. 10A-B and 12A-B and generally includes flexible supports 232 extending from a base 234 defining an opening 236 therein to receive the probe 220. The navigation device probe adapter 220 may be inserted into the obturator 222. The locking member 226 may lock the stylet 253 in place via a thumb screw. An obturator base 240 may include a biasing member such as a spring or a leaf spring to allow for the obturator 222 to compress. When the navigation device probe adapter 220 is inserted into the obturator 222, the navigation device probe adapter 220 forces the obturator 222 open to allow for the distal end of the soft tissue cuff (not shown in FIGS. 8 and 9) to be placed onto the obturator 222. The navigation device probe adapter 220 may maintain the obturator 222 in an expanded state. The soft tissue cuff 200 may be maintained at a distal end of the obturator 222.

When assembled with the navigation device probe adapter 220, the stylet 253 may extend into the opening 236 of the obturator 222 up until a drill point 252. The distance between the distal tip 230 of the stylet 253 and an apex 242 of the obturator 222 may be approximately 15 mm. However, the distance may be greater or lesser than 15 mm.

Once the navigation device probe adapter 220, obturator 222 and soft tissue cuff 200 are assembled, the assembly 218 may be placed at the surgical site. Placement to the desired location within the tissue may occur with or without navigation. Subsequently, once placed, the navigation device probe adapter 220 may be removed from the obturator 222. The obturator 222 may then collapse and be removed. Once the obturator 222 collapses, the soft tissue cuff 200 reforms to its relaxed state, which facilities the removal of the obturator 222 in the collapsed state while leaving the soft tissue cuff 200 at the surgical site creating an access entry point for the surgical tool, as described above.

FIG. 10A illustrates a perspective view of an obturator 222 in an expanded state. FIG. 10B illustrates a perspective view of the obturator 222 of FIG. 10A in a collapsed state. As explained above, the obturator 222 includes a pair of flexible supports 232 attached at the apex 242. The apex 242 is configured to act as a hinge between the flexible supports 232 so that the supports 232 may move at the apex 242 with respect to one another allowing the obturator 222 to move between a collapsed and expanded state. The supports 232 may form a wedge-like shape to align with the sulcus of the surgical site.

The probe base 234 may form two halves 244, each half 244 carrying an opposite end of one of the supports 232. The two halves 244 configured to move as the supports 232 move to be closer or further from the other and correspondingly increasing the footprint of the obturator to ease frictional forces during the deployment of the cuff 200 from the obturator. In the collapsed state, as illustrated in FIG. 10B, the supports 232 and the base 234 may form an opening 236 that is smaller than the opening 236 in the expanded state.

As best shown in FIG. 10A, the obturator 222 may include a pair of biasing members 238 or springs 238 arranged on an underside of the base 234 and connecting each of the halves 244 of the base 234. The springs 238 may be arranged parallel to one another and be configured to bias the base 234 to the expanded state. The springs 238, in addition to the position of the probe within the obturator 222, may keep the obturator 222 in the expanded state until the clinician is ready to deploy the soft tissue cuff 200 from the obturator 222. While a pair of springs 238 is described, more or less springs, and springs in nonparallel arrangements, may be recognized.

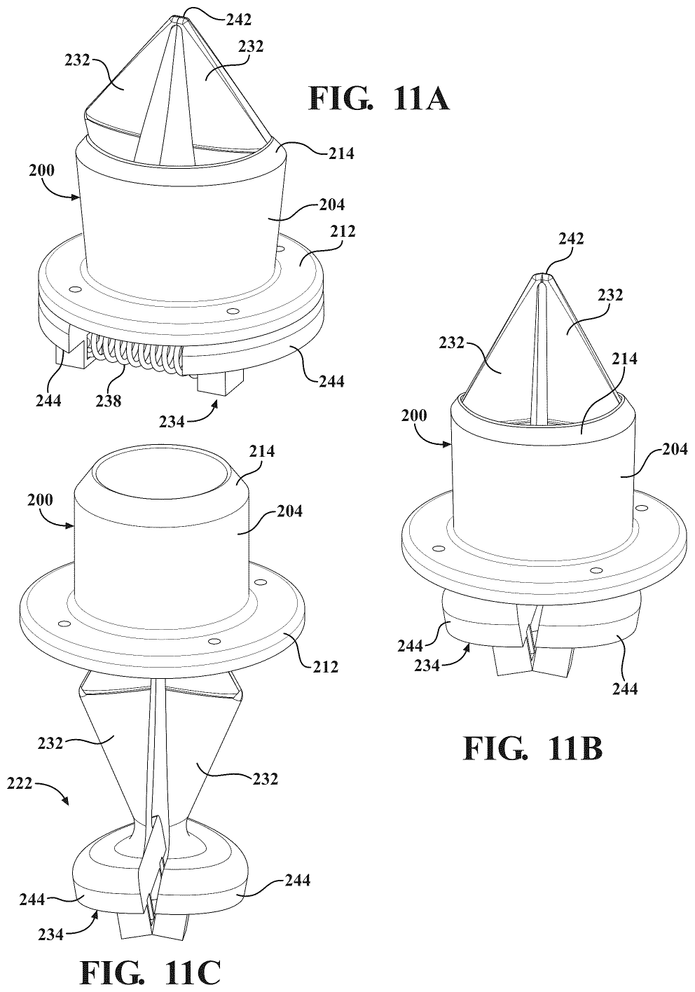

FIG. 11A illustrates a perspective view of the obturator 222 of FIG. 10A in an expanded state within the soft tissue cuff 200. The soft tissue cuff 200 is configured to fit over the obturator 222 and fit to the obturator's shape. This state may be achieved when the distal tip 230 of the navigation device probe adapter 220 is arranged within the obturator 222. The navigation device probe adapter 220 may be received by the obturator 222 and may force the supports 232 outward at the apex 242. The assembly may be inserted at the surgical site in the expanded state.

FIG. 11B illustrates a perspective view of the obturator 222 of FIG. 10A in a collapsed state with the soft tissue cuff 200 arranged thereon. Once the assembly has been inserted at the surgical site, the navigation device probe adapter 220 may be removed from the obturator 222. This may cause the supports 232 of the obturator 222 and the halves 244 of the base 212 to retract toward each other due to the compression by the soft tissue cuff 200, thus causing the obturator 222 to resume the collapsed state.

FIG. 11C illustrates a perspective view of the obturator 222 of FIG. 11A upon removal from the soft tissue cuff 200. The obturator 222 may contract, allowing the navigational device probe adapter 220 to be removed from the soft tissue cuff 200. The soft tissue cuff 200 remains at the surgical site to provide access for the surgical tools.

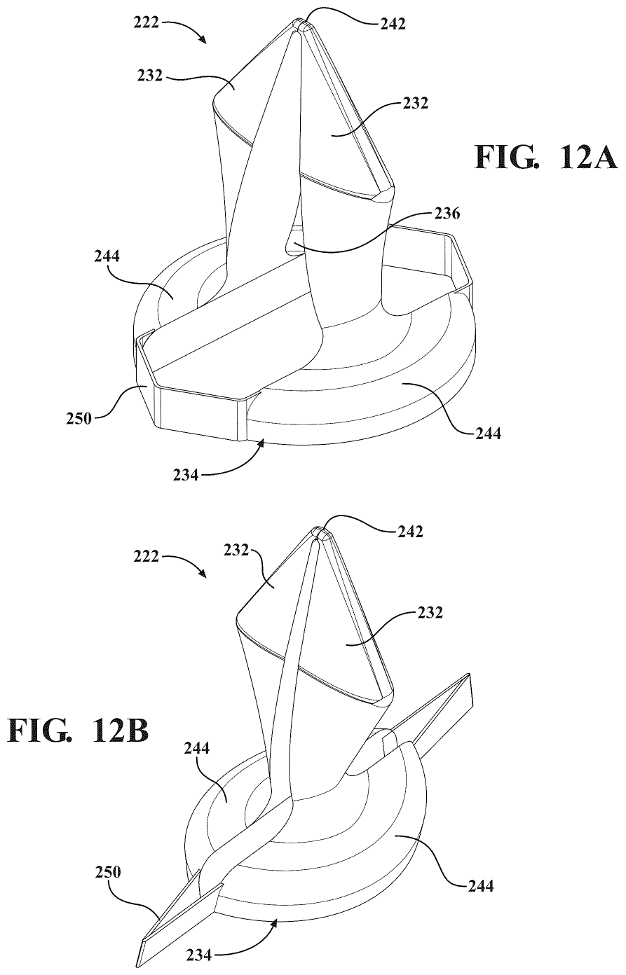

FIG. 12A illustrates a perspective view of another obturator 222 in an expanded state. In this example, instead of a biasing member 238, the obturator 222 may include a pair of leaf springs 250 to bias the obturator 222 into the expanded state. The leaf springs 250 may be formed of polycarbonate. The soft tissue cuff 200 is configured to fit over the obturator 222 and to generally fit the shape of the obturator 222. This state may be achieved when the distal tip 230 of the navigation device probe adapter 220 is arranged within the obturator 222. Specifically, the distal tip 230 may be received by the opening in the obturator 222 up to the drill point 252, or a predefined distance from the apex 242 of the obturator 222 and may force the supports 232 outward. The assembly 218 may be inserted at the surgical site in the expanded state.

FIG. 12B illustrates a perspective view of the obturator 222 of Figure I IA in a collapsed state with the soft tissue cuff 200 arranged thereon. Once the assembly has been inserted at the surgical site, the navigational device probe adapter 220 may be removed from the obturator 222. This may cause the supports 232 of the obturator 222 and the halves 244 of the base 212 to retract toward each other due to the compression by the soft tissue cuff 200, causing the obturator 222 to resume the collapsed state.

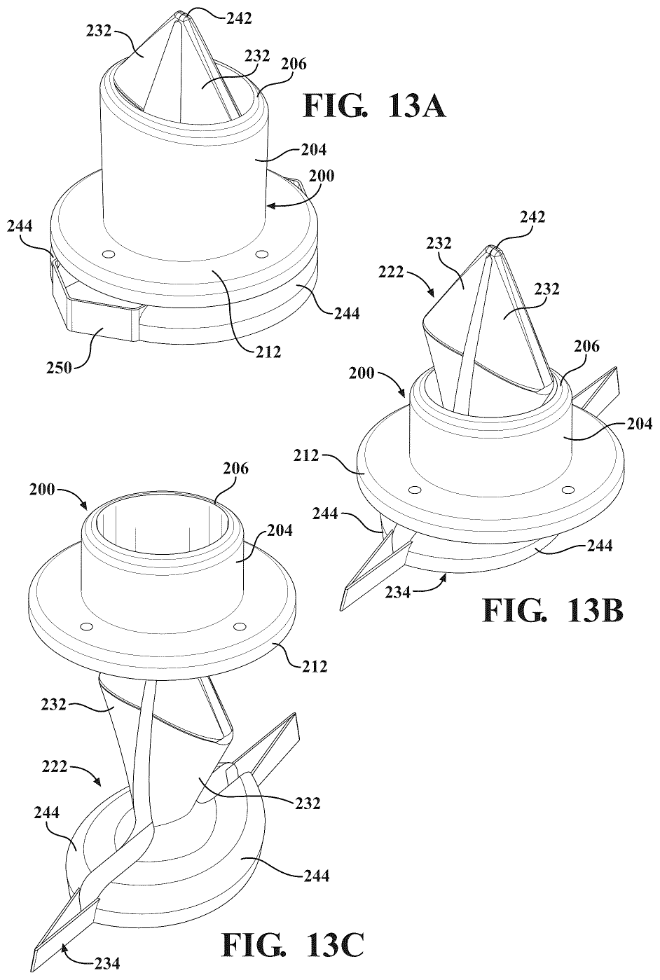

FIG. 13A illustrates a perspective view of the obturator 222 of FIG. 12A in an expanded state within the soft tissue cuff 200. The soft tissue cuff 200 is configured to fit over the obturator 222 and fit to the general shape of the obturator 222. This state may be achieved when the distal tip 230 of the navigation device probe adapter 220 is arranged within the obturator 222. The navigation device probe adapter 220 may be received by the obturator 222 and may force the supports 232 outward at the apex 242. The assembly may be inserted at the surgical site in the expanded state.

FIG. 13B illustrates a perspective view of the obturator 222 of FIG. 12A in a collapsed state within the soft tissue cuff 200 arranged thereon. Once the assembly has been inserted at the surgical site, the stylet 253 (e.g., the navigation probe 253) may be removed from the obturator 222 and the navigation device probe adapter 220. This may cause the supports 232 of the obturator 222 and the halves 244 of the base 212 to retract toward each other due to the compression by the soft tissue cuff 200, thus causing the obturator 222 to resume the collapsed state.

FIG. 13C illustrates a perspective view of the obturator 222 of FIG. 12A upon removal from the soft tissue cuff 200. The obturator 222 may contract, allowing the obturator 222 to be removed from the soft tissue cuff 200. The soft tissue cuff 200 remains at the surgical site to provide access for the surgical tools.

Accordingly, a soft tissue or cortical cuff is disclosed herein to allow access and inspection to tissues over lying and below a cortical rim. The cortical cuff may be flexible so as to be foldable during installation. The soft tissue cuff may resume a relaxed, circular shape after installation whereby the soft tissue cuff may gently grip the cortex to create a stable fit therein and to prevent the soft tissue cuff from moving. At the end of a procedure, during clean up, the soft tissue cuff may absorb forces thereon created by a surgical tool. The forces may dissipate through the soft tissue cuff's radial shape in an effort to avoid a single compressive force to the surrounding tissue.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this disclosure have been explained and illustrated in exemplary embodiments.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the disclosure should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. A soft tissue cuff apparatus for subcortical surgery of the brain, comprising:
a cuff body forming a ring-like shape in an expanded position, the cuff body being formed of a flexible material,
the cuff body extending between a first lip and a second lip and forming a concave outer periphery where, during use, the cuff body is configured to contact and cradle at least a portion of the cortex and where the lips are configured to form a hold on the cortex such that the soft tissue cuff apparatus creates a stable fit within the cortex to form a surgical corridor and allow access to a surgical site under the cortex without subjecting the surrounding soft tissue to further manipulation.

2. The soft tissue cuff apparatus of claim 1, wherein the cuff body is formed of a flexible elastomer including at least one of silicone, rubber, urethane, or PVC.

3. The soft tissue cuff apparatus of claim 1, wherein the cuff body is movable between a folded position during insertion into the surgical corridor and the expanded position forming the stable fit within the cortex.

4. The soft tissue cuff apparatus of claim 1, wherein the cuff body forms an inner surface forming an inner diameter and concave outer periphery formed with the first and second lips defines a radius about the concavity, with a ratio range of the radius to inner diameter being 1:5 to 1:3.

5. A soft tissue cuff apparatus for subcortical surgery of the brain, comprising:
a cuff body forming a cylinder-like shape in a resting state, the cuff body being formed of a flexible material,
the cuff body extending between a distal end and a base opposite the distal end, the base having a circular shape, wherein the cuff body is configured to receive an obturator, wherein the cuff body conforms to the shape of the obturator during insertion of the cuff body at the surgical site and reverts to the resting state not conforming to the shape of the obturator and releasing from the obturator upon collapse of the obturator caused by a removal of a navigation device probe adapter within the obturator during placement of the cuff body at the surgical site.

6. The soft tissue cuff apparatus of claim 1, wherein the flexible material of the cuff body is an elastomer including at least one of silicone, rubber, urethane, or PVC.

7. A soft tissue cuff assembly, comprising:
a soft tissue cuff having a cuff body forming a cylindrical shape and extending between a distal end and a base;
an obturator configured to receive the soft tissue cuff and having a navigation device probe adapter configured to receive a navigation probe for placement of the soft tissue cuff at a surgical site.

8. The assembly of claim 7, wherein the obturator includes a base forming two halves and configured to move relative to the other half between an expanded state and a collapsed state, where in the expanded state, the soft tissue cuff conforms to the shape of the obturator.

9. The assembly of claim 8, wherein the obturator includes a pair of supports, one extending from each half of the base and being hingedly connected at an opposite end of the halves to expand an opening between the supports when the probe adapter is in the expanded state.

10. The assembly of claim 9, wherein the supports form a wedge-like shape.

11. The assembly of claim 8, wherein the halves of the base are connected and biased away from each other by a biasing member.

12. The assembly of claim 11, wherein the biasing member is a pair of springs.

13. The assembly of claim 11, wherein the biasing member is a pair of leaf springs.

14. The assembly of claim 9, wherein the navigation device probe adapter is configured to be received by the obturator and expand the obturator within the soft tissue cuff and collapse the obturator when removed from the obturator to place the soft tissue cuff at the surgical site.

15. The assembly of claim 9, wherein the soft tissue cuff is malleable.

16. The assembly of claim 9, wherein the soft tissue cuff is pliable.

17. The assembly of claim 9, further comprising a navigation device probe adapter configured to be received by the obturator thereby extending the obturator to an expanded state such that the soft tissue cuff conforms to the shape of the obturator, and upon removal the navigation probe adapter from the obturator, collapsing the obturator to a collapsed state; wherein the soft tissue cuff is configured to be released from the collapsed obturator and retain a resting shape.

18. The assembly of claim 17, wherein the navigation device probe adapter is configured to retain a navigation probe, with the navigation probe extending from the navigation probe adapter towards an apex and wherein the assembly is configured to retain the navigation probe such that a distal end of the navigation probe is maintained at a fixed distance from an apex defined as the hinged connection point of each half of the base of the obturator.
